SEQUENCE LISTING (15 sequences)

SEQ ID NO: 1 - protein, 393 aa, Tsukamurella paurometabola
MIAPQIRLGEPDTTPLPDPLWHGAPPAPPTTVAVIESLATGSPAQAHGQSVSAERVAARFADPIQAERIRRVYANTAVATRHLAIDPLSDDFADFSARPDTIRQRMDLYFEHAAPLAIETARRALGAVDATEVGQLIFVTSTGFLAPGVDVAVTRSLGLPASTSRVVINFMGCAAAMNALRVASDFVRAHPDRKSLLVCLELSSVNAVFAGDPNDVVISSLFADGCGAALIGASEVGHPLPAGHIVVRDTFAHLLDDAEDGIVLGVNANGITCELAHSLPRYILDGVSPVIDGVLARNGLGREDVAHWAIHPGGPKIIESASAALGLPRSASQTSWAVLAEHGNMLSVSLLFVLERLLGARAAGGTQETGLAFSFAPGVTVEGFLFDVVGGPS

SEQ ID NO: 2 - protein, 412 aa, Tsukamurella tyrosinosolvens
MTMNAGSQGAPALDVPAPVLPAPGTWLGAPPAPPTSVAVIESLATGSPAQTYGQAESADRVAARFDDPRQAERIRRVYAKTRVDERHLAIDPLTPEFAEFSTRPDTVRERMDLFYEHAAPLAVDTARRALGVGTEHEFDPADVGQLVFVTSTGFLAPGVDVAVIRALGLAPQTSRVVINFMGCAAAMNALRVSTDYVRAHPDRKSLMICLELSSVNAVFSGDPNDVVISSLFADGCGAAVIGASEVGHPLPGGRIVVRDTFTHLLDGAEDGIVLGVNANGITCELAESLPQYIVDGVAPVIDEVLGRNDLGREAVAHWAIHPGGPKIIESAATALGLPDEASRTSWDVLAEHGNMLSVSLLFVLERLLAQVADGAATQPDGAPTTGMAFSFAPGVTVEGFLFDVVTGDQPGE

SEQ ID NO: 3 - protein, 387 aa, Tsukamurella pseudospumae
MAAPELPGTSVWRGAPPAPPTSVAVIESLATGSPSQAYDQAESADRVASRFDDPRQAERIRRVYAKTRVAERHLAIDPLTPEFAAFSTRPDTIRERMDLFFEHAAPLAIDTARRALGTVDPADVGQLVFVTSTGFLAPGVDVAVIRALGLSPGTSRVVINFMGCAAAMNALRVSTDYVRAHPDRKSLMICLELSSVNAVFSGDPNDVVISSLFADGCGAALIGASEVGHPLPAGNIVVRDTFSHLLDGAEDGIVLGVNADGITCELAESLPQYIVDGVAPVVDGVLRRNGLDRDAVAHWAIHPGGPKIIESASTALGLPDEASRLSWDVLAGHGNMLSVSLLFVLERLLAQVASDGADGAPSTGMAFSFAPGVTVEGFLFDVVTTGE

SEQ ID NO: 4 - protein, 390 aa, Tsukamurella pulmonis
MNTTEQQSVLPQQAWLGAPPAPPTSVAVIESLATSSPTQTYGQAESADRVAARFDDPRQAERIRRVYAKTRVTERHLAIDPLTPEFAEFSARPDTVRERMDLFFEHAAPLAIETARRALGDNAATDIGQLVFVTSTGFLAPGVDVAVIRALGLAPQTSRVVINFMGCAAAMNALRVSTDYVRAQPDRKSLMICLELSSVNAVFSGDPNDVVISSLFADGCGAAVIGASEVGHPLPSGQIVVRDTFTHLLDGAEDGIVLGVNANGITCELAESLPQYIVNGVAPVVDAVLDRNGLGREAVAHWAIHPGGPKIIESAAAALGLPDDASRLSWDVLAEHGNMLSVSLLFVLERLRAQVASDGADGAPSTGMAFSFAPGVTVEGFLFDVVTTGE

SEQ ID NO: 5 - protein, 397 aa, Tsukamurella sp.
  source 1..397: note = Strain: 1534
MNIQDRSTVAPDSPALGFPPAPPTSVAVIESLATGSPSGVHAQAESADRVASRFDDPAQAERIRRVYTKTRVARRHLAIDPLDEDFAAFSARPDTIRERMDLFAEHASPLAVDTARRALGAVDPADVGQLVFVTSTGFLAPGVDVAIVRALGLPATTSRVIVNFMGCAAAMNALRVASDFVRAHPDRKSLMVCLELSSVNAVFSGDPNDVVISSLFADGCGAAVIGASEVGHPLPGGSIVVRDTFAHLLDGAEDGIVLGVNANGITCELAESLPRYIVDGVRPVIDGVLARNGLGHADVAHWAIHPGGPKIIESASAALGLPAAASATSWQVLAEHGNMLSVSLLFVLERMLSGLPDGAPSTGMAFSFAPGVTVEGFLFDIVRDGAPRPGALGGRPA

SEQ ID NO: 6 - protein, 400 aa, Nocardia farcinica
MSITVDEGGARPATEPRQRIHPDLGHAHTPMPPAPPVTIGVVEGIATGSPAQIVDQAEAAERVAALFTDPAQRARIARVYEKTRIETRRMAVDPTAPEFRSFSRQPGTLRERMNLFYRHAAPLAVDVAGRALADSGAAAADIGLLVFVTSTGFIAPGVDVAVLRELGLVPTVGRVVVNFMGCAAAMNGIRTGVDYVRAHPDKKALVVCLELSSVNAVFADDVNDVIIHSLFGDGCGAVVLGASQARQPLAPGRVVVRDSFSHLFDAAEDGIVLGVDHNGITCELSENLPRYIYRGVDPVVREVLARNGLRKSDIDLWAIHPGGPKIIEESVRSLGLSPEQAAPSWDVLARHGNMLSVSLIFVLEQMIAQSATAEPISTGVAFSFAPGVTLEGLIFDIIRR

SEQ ID NO: 7 - protein, 393 aa, Mycobacterium marinum
MSTAAEGGAIRRAGHEPRYDLAQLPPAPPTTVAVIEGMATGAPQRVVAQADAAARVSELFVDPQQRERISRIYDKTRIDTRRMAVDPLDDEFDEFRREPATIRDRMNLFYQHAVPLAVDVAARALDGLPYAPDEIGQLVFVTSTGFIAPGVDVEIVKQLGLPRSISRVVVNFMGCAAAMNAIRTATNYVRAHPSMKALVVCIELSSVNAVFADDINDVVIHSLFGDGCGALVIGASQVQQPLPAGNVVIRSSFSQLLDDSEDGIVLGVNHDGITCELSENLPSYIYRSVDPVVAEMLRDNGLSKADIDLWAIHPGGPKIIEQSARSLGIPVGRAVQSWDVLAQFGNMLSVSLIFVLEMMVAQAESDKPISTGVAFAFAPGVTVEGMLFDIIRR

SEQ ID NO: 8 - protein, 395 aa, Mycobacterium kansasii
MSSAADGGAPVADVPGYEPHYDLAQLPPAPPTTVAVIEGMATGVPQRVVRQSDAAARVAQMFVDPQQRERVSRVYAKTRIDTRRMAVNPLDAEFDAFRREPATIRDRMSLFYRHAVPLAVEVTRRALAGLSYGADEIGLLVFVTSTGFVAPGVDVAIVKELGLSRAISRVVVNFMGCAAAMNAIRTATNYVRAHPAMKALVVCIELCSVNAVFADNVKDVVIHSLFGDGCAALVIGASQVQQQLPAGSVVIRSNFSQLLDDAEDGIVLGVNHDGITCELSENLPDYIYRGVAPVVANVLYDNGLQQSDIDLWAIHPGGPKIIEQSVRSLGIGVECAAPSWDVLARYGNMLSVSLIFVLEMMVQQAESEKPLSTGVAFAFAPGVTVEGMLFDIVRR

SEQ ID NO: 9 - protein, 334 aa, Mycobacterium tuberculosis
MNVSAESGAPRRAGQRHEVGLAQLPPAPPTTVAVIEGLATGTPRRVVNQSDAADRVAELFLDPGQRERIPRVYQKSRITTRRMAVDPLDAKFDVFRREPATIRDRMHLFYEHAVPLAVDVSKRALAGLPYRAAEIGLLVLATSTGFIAPGVDVAIVKELGLSPSFGDGCAALVIGASQVQEKLEPGKVVVRSSFSQLLDNTEDGIVLGVNHNGITCELSENLPGYIFSGVAPVVTEMLWDNGLQISDIDLWAIHPGGPKIIEQSVRSLGISAELAAQSWDVLARFGNMLSVSLIFVLETMVQQAESAKAISTGVAFAFGPGVTVEGMLFDIIRR

SEQ ID NO: 10 - protein, 370 aa, Gordonia hydrophobica
MPAPVTTVAVIEAVATGAPATVHPQTRAAEQVAELYDDPALQERIRRLYRNTRVQTRHLAVDPMTPEFQEFSSRPATVRTRMNDYFHHAVPLAVDVARRALAGVTDPATEIGQIIFVTSTGFIAPGVDVAVITELGLAPTVHRVIINFMGCAAAINGISTATDHVRANPDSRALLICLELSSVNAVFGADPVELVTHSLFGDGCGAMLIGASPVGRRLAPGQLVVRDTFSHLFHDTGDGIVLGVNDDGITCELAQELPSYIRRGVGPAIDAALNRSRLRRDDIAHWAIHPGGPAIIEQSVAALDLPPDRAATSWEVLAEYGNMLSVSLVFVLEKLIAAGAHGRGQAPETGVAFSFAPGVALEGMLFDLVC

SEQ ID NO: 11 - protein, 349 aa, Pseudomonas fluorescens
  source 1..349: note = Strain: Pf-5
MSTLCLPHVMFPQHKITQQQMVDHLENLHADHPRMALAKRMIANTEVNERHLVLPIDELAVHTGFTHRSIVYEREARQMSSAAARQAIENAGLQISDIRMVIVTSCTGFMMPSLTAHLINDLALPTSTVQLPIAQLGCVAGAAAINRANDFARLDARNHVLIVSLEFSSLCYQPDDTKLHAFISAALFGDAVSACVLRADDQAGGFKIKKTESYFLPKSEHYIKYDVKDTGFHFTLDKAVMNSIKDVAPVMERLNYESFEQNCAHNDFFIFHTGGRKILDELVMHLDLASNRVSQSRSSLSEAGNIASVVVFDVLKRQFDSNLNRGDIGLLAAFGPGFTAEMAVGEWTA

SEQ ID NO: 12 - protein, 381 aa, Ectocarpus siliculosus
MSKDEQTVYPVIAGMAIGNPQYRCTQNEALAVASKCPGLESIKPVLERIYGNSRIGSRYFAVPDFTPGRAAKGDPLFYPADGSYQVPVDVRLDKFKEKAVPLVSDVARRAIKEAGLNVEDISKLVVVSSTGFLGPGLDCELIKNLGLTRSVDRTLIGFMGCAAAMNGFRNANDYVTANPGKYALMICVELSSVHTTFDDNINDAILHAIFADGCAAAVLKGARKSECPKGTLAIVDNHAWLMEGTEDGITLAIKPNGITCTLSKFLPQYIAKNIAFFADGFLKKHKLGRDDVDFWCVHPGGRRIIEEAQNGLGLSEEQTADSWAVLGEYGNMLSPSVMFVLSRVFKRHNAALAQGKPGYQTGMAFSFSPGVGAEGILLRQI

SEQ ID NO: 13 - DNA, 598 bp, Saccharomyces cerevisiae
  source 1..598: mol_type = unassigned DNA
tatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacaca

SEQ ID NO: 14 - DNA, 998 bp, Saccharomyces cerevisiae
  source 1..998: mol_type = unassigned DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaata

SEQ ID NO: 15 - DNA, 300 bp, Saccharomyces cerevisiae
  source 1..300: mol_type = unassigned DNA
gaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgcttactttcttcttttttgccgttttgacttggacctctggtttgctatttccttacaatctttgctacaat

(19) United States
(12) Patent Application Publication
(10) Pub. No.: US 2025/0011821 A1
(43) Pub. Date: Jan. 9, 2025

(54) USE OF TYPE III POLYKETIDE SYNTHASES FROM BACTERIA AS PHLOROGLUCINOL SYNTHASES

(71) Applicant: COMPAGNIE GENERALE DES ETABLISSEMENTS MICHELIN, Clermont-Ferrand (FR)

(72) Inventors: VINCENT LAFAQUIERE, Clermont-Ferrand (FR); ODILE RAMAEN, Ablis (FR); DOMINIQUE LOUIS, Forges Les Bains (FR)

(21) Appl. No.: 18/591,697

(22) Filed: Feb. 29, 2024

Related U.S. Application Data
(62) Division of application No. 16/627,712, filed on Dec. 30, 2019, now Pat. No. 11,920,178.

(30) Foreign Application Priority Data
Jun. 30, 2017 (FR) 1756106

(51) Int. Cl. C12P 7/22; C12N 9/10

(52) U.S. Cl. CPC C12P 7/22; C12N 9/1029; C12Y 203/01

(57) ABSTRACT

Methods for producing phloroglucinol as well as methods of using type III polyketide synthases of bacteria, such as actinomycete bacteria, as phloroglucinol synthases, are described herein. In addition, isolated nucleic acid molecules encoding these type III polyketide synthases, and vectors and the host cells comprising such nucleic acid molecules are described.

The present application is a divisional application of U.S. patent application Ser. No. 16/627,712, which is a national stage entry of PCT Application No. PCT/FR2018/051619, filed on Jun. 29, 2018, which claims the benefit of foreign priority of French Application No. 1756106 filed on Jun. 30, 2017, the entirety of each of which is incorporated herein by reference for all purposes.

The contents of the electronic sequence listing (18657_589894_SL.xml; Size: 20,911 bytes; and Date of Creation: Aug. 22, 2024) is herein incorporated by reference in its entirety.

### FIELD OF THE INVENTION

The present invention lies in the fields of microbial biochemistry and more particularly in the field of the synthesis of phloroglucinol by microbial enzymes. It relates to the use of type III polyketide synthases from bacteria, in particular from actinomycete bacteria, as phloroglucinol synthases.

### BACKGROUND

Phloroglucinol is an aromatic organic compound used in particular in the production of pharmaceutical products and explosives.

Phloroglucinol synthesis is catalysed by type III polyketide synthases known as phloroglucinol synthases. Phloroglucinol synthases carry out the condensation of three malonyl-CoA molecules so as to form a phloroglucinol molecule according to the following reaction scheme (Reaction I):

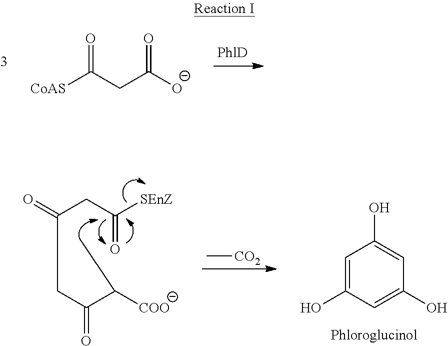

Numerous oligomers can subsequently be synthesized from phloroglucinol, such as phlorotannins. Phlorotannins include in particular fucols, phloretols and fucophloretols, which are phloroglucinol-derived products that make up the wall of brown algae. In addition, various protective activities of brown algae have also been attributed to phlorotannins.

At the current time, phloroglucinol synthesis has been described only in Gram− Pseudomonas fluorescens bacteria (Achkar et al., 2005; Zha et al., 2006) and in the brown alga Ectocarpus siliculosus (Meslet-Cladière et al., 2013). The phloroglucinol synthase enzyme involved in phloroglucinol synthesis has been identified in these two species. They are the only two phloroglucinol synthases identified and characterized to date.

In Pseudomonas fluorescens, the phloroglucinol synthase is encoded by the PHLD gene (Achkar et al., 2005; Zha et al., 2006).

In Ectocarpus siliculosus, the phloroglucinol synthase is encoded by the PKS1 gene (Meslet-Cladière et al., 2013).

It has been possible to demonstrate PHLD phloroglucinol synthase activity in Escherichia coli expressing a heterologous PHLD gene (Achkar et al., 2005). This activity has been confirmed in vitro, by means of small-scale enzymatic tests carried out with a recombinant PHLD expressed and purified from Escherichia coli cultures (Zha et al., 2006).

PKS1 phloroglucinol synthase activity has been demonstrated in vitro, from recombinant PKS1 expressed and purified in Escherichia coli and from cell extracts of E. siliculosus (Meslet-Cladière et al., 2013, WO 2013/045510).

However, the PHLD and PKS1 enzymes exhibit low enzymatic activities. In addition, the possibility of synthesizing phloroglucinol in vitro on a large scale using these enzymes has never been proved. Finally, the phloroglucinol synthases used in these studies were produced by E. coli or P. fluorescens bacteria. The activity of these enzymes when they are produced by eukaryotics, such as yeasts or insect or mammalian cells, is thus unknown. However, the eukaryotic systems may be advantageous, in particular for large-scale productions. They in fact make it possible to obtain enzymes which can be modified at the post-translational level.

Thus, there is still the need to identify new phloroglucinol synthases which have a high phloroglucinol synthesis enzymatic activity in vitro or in vivo, which are suitable for industrial-scale production and which can be produced by eukaryotic systems.

The exact functional characterization of a polyketide synthase is, however, complicated by the fact that this class of enzymes brings together proteins which have large sequence similarities, whereas they can catalyse substantially dissimilar reactions and recognize entirely different substrates.

Despite these difficulties, the present inventors have been able to identify polypeptides which are present in the bacteria and have phloroglucinol synthase activity. Thus, new phloroglucinol synthases have been identified, in particular in Gram+ bacteria. They constitute the first example of phloroglucinol synthases in bacteria of this type. The inventors demonstrate here that these new phloroglucinol synthases exhibit a high phloroglucinol synthesis activity. They are thus suitable for industrial-scale production. Furthermore, they are functional when they are produced in a eukaryotic system.

### SUMMARY OF THE INVENTION

In the context of the present invention, the inventors have demonstrated, entirely surprisingly, that bacteria, in addition to Pseudomonas fluorescens, contain in their genome a gene encoding a type III polyketide synthase having a functional phloroglucinol synthase activity.

The present invention thus relates to polypeptides chosen from type III polyketide synthases from bacteria, in particular type III polyketide synthases from gram+ bacteria, in particular the type III polyketide synthases from actinomycete bacteria, which can be used as phloroglucinol synthases.

The present invention also relates to isolated nucleic acid molecules encoding phloroglucinol synthases from bacteria, in particular encoding phloroglucinol synthases from gram+ bacteria, in particular for type III polyketide synthases from actinomycete bacteria, and also to the phloroglucinol synthases thus encoded.

The invention also relates to vectors comprising at least one isolated nucleic acid molecule encoding such a phloroglucinol synthase.

The invention also relates to host cells comprising at least one isolated nucleic acid molecule or at least one vector according to the invention.

The invention also relates to methods for producing a functional phloroglucinol synthase.

The invention also relates to methods for producing phloroglucinol.

### DESCRIPTION OF THE FIGURES

FIG. 1 presents the protein sequence alignment of the identified candidate enzymes. The alignment of the ten selected enzymes with the enzyme PKS1.Es (PHLD.Es) was carried out using the Clustal W software.

FIG. 2 presents an example of phloroglucinol/resorcinol separation on a propyl-pentafluorophenyl (PFP) column.

FIG. 3 shows an example of the structure of a gene unit constructed for a given candidate (PhlD.ii), making it possible to express the PHLD genes in the yeast Saccharomyces cerevisiae.

FIGS. 4A, 4B, 4C, and 4D show the levels of phloroglucinol production in the yeast strains expressing the various candidate genes PHLD.ii and the control PHLD.Pf and PKS1.Es genes (several copies) under the control of the ADH2 promoter, after 48 hours of culture in a 24-well plate in the presence of 20 g.l⁻¹ of ethanol as carbon source at 30° C. In particular, FIG. 4A is a summary of the various data measured. FIG. 4B shown optical densities (OD) of the various cultures measured at 600 nm (OD₆₀₀), indicating the level of growth of each strain. FIG. 4C shows the level of phloroglucinol production (in mg.l⁻¹) measured in the culture medium. FIG. 4D shows the level of phloroglucinol production (in mg.l⁻¹) standardized relative to the number of copies of genes integrated into the genome.

FIG. 5 shows the structure of the gene constructs integrated into the genome of the yeast at the JLP1 locus. The gene encoding each PHLD/PKS1 is under the control of the pADH2 promoter or of the pCCW12 promoter.

FIGS. 6A, 6B, and 6C show the levels of phloroglucinol production in the yeast strains expressing the various PHLD.ii genes (only 1 copy) under the control of the ADH2 promoter after 48 hours of culture in a 24-well plate in the presence of 20 g.l⁻¹ of ethanol as carbon source at 30° C. In particular, FIG. 6A is a summary of the various data measured. FIG. 6B shows optical densities (OD) of the various cultures measured at 600 nm (OD₆₀₀), indicating the level of growth of each strain. FIG. 6C shows the level of phloroglucinol production (in mg.l⁻¹) measured in the culture medium.

FIGS. 7A, 7B, and 7C show the levels of phloroglucinol production in the yeast strains expressing the various PHLD genes (only 1 copy) under the control of the CCW12 promoter after 48 hours of culture in a 24-well plate in the presence of 20 g.l⁻¹ of glucose as carbon source at 30° C. FIG. 7A is a summary of the various data measured. FIG. 7B shows optical densities (OD) of the various cultures measured at 600 nm (OD₆₀₀), indicating the level of growth of each strain. FIG. 7C shows the level of phloroglucinol production (in mg.l⁻¹) measured in the culture medium.

### DETAILED DESCRIPTION OF THE INVENTION

### Definitions

The term “type III polyketide synthase” is intended to mean a multifunctional enzyme or an enzymatic complex producing polyketides and which does not use an acyl carrier protein (or ACP) domain.

The term “polyketide” is intended to mean a large family of secondary metabolites in bacteria, mycetes, plants and certain animal lines which originate from the iterative condensation of acetyl or malonyl subunits by polyketide-synthase enzymes. Polyketides also serve as starting materials for the production of a wide range of natural and semi-synthetic products.

The term “phloroglucinol” is intended to mean an aromatic organic compound benzene-1,3,5-triol having the following chemical formula (formula I):

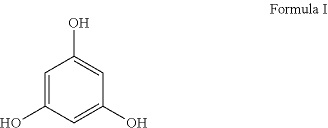

The term “phloroglucinol synthase” is intended to mean a multifunctional enzyme or an enzymatic complex which belongs to the family of type III polyketide synthases and which catalyses phloroglucinol synthesis. A phloroglucinol synthase catalyses the condensation of three malonyl-CoA molecules so as to form a phloroglucinol molecule.

The term “enzymatic activity” or “catalytic activity” or else “activity” of an enzyme is intended to mean the efficiency of an enzyme to convert a substrate into a product in a given environment. The efficiency of the enzyme takes into account here the rate of conversion of the substrate into a product by the enzyme and the degree of conversion of the substrate into a product by the enzyme. The expression “degree of conversion of the substrate into a product by the enzyme” is intended to mean here the ratio between the amount of final product obtained relative to the initial amount of substrate for a defined amount of enzyme. For example, for the purposes of the invention, an enzymatic activity can be expressed as an amount of phloroglucinol produced in a given volume (in g/l).

The term “bacterium” is intended to mean a microscopic and prokaryotic organism present in all media.

The term “Gram+ bacterium” or “Gram-positive bacterium” is intended to mean a bacterium which is positive upon Gram staining (that is to say which retains the gentian violet, also known as crystal violet, and remains mauve-violet-coloured or blue-dark purple-coloured).

The term “actinomycete bacterium” or “Gram+ actinomycete bacterium” or “Gram-positive actinomycete bacterium” is intended to mean a bacterium belonging to the order Actinomycetales in the classification of actinobacteria, which is positive with respect to Gram staining. Actinomycetes are bacteria, the growth of which give rise to colonies consisting of hyphae, that is to say filaments which radiate, by centrifugal growth, all around the microorganism which gave rise to them.

The term “Tsukamurella sp.” is intended to mean a rod-shaped, necessarily aerobic, non-sporulating actinomycete bacterium of the Tsukamurella genus. The Tsukamurella genus comprises in particular the species Tsukamurella paurometabola (also referred to as Tp hereinafter), Tsukamurella tyrosinosolvens (also referred to as Tt hereinafter), Tsukamurella pseudospumae (also referred to as Tps hereinafter), Tsukamurella pulmonis (also referred to as Tpu hereinafter) and Tsukamurella sp. 1534 (also referred to as Tsp hereinafter).

The term “Nocardia sp.” is intended to mean a filamentous actinomycete bacterium of the Nocardia genus. The Nocardia genus comprises in particular the species Nocardia farcinica (also referred to as Nf hereinafter).

The term “Mycobacterium sp.” is intended to mean a non-sporulating, aerobic actinomycete bacterium, in the shape of bacilli, of the Mycobacterium genus. The Mycobacterium genus comprises in particular the species Mycobacterium marinum, Mycobacterium kansasii and Mycobacterium tuberculosis (also respectively referred to as Mma, Mk and Mt hereinafter).

The term “Gordonia sp.” is intended to mean an actinomycete bacterium of the Gordonia genus. The Gordonia genus comprises in particular the species Gordonia hydrophobica (also referred to as Gh hereinafter).

The term “Pseudomonas sp.” is intended to mean a Gram-negative (Gram−) bacterium, which does not form spores (or non-sporulating), which is in the form of a bacillus and which is necessarily aerobic, of the Pseudomonas genus. The Pseudomonas genus comprises in particular the species Pseudomonas fluorescens (also known as Pf hereinafter).

The term “Ectocarpus sp.” is intended to mean an alga of the Ectocarpus genus, of the class of brown algae, belonging to the family Ectocarpaceae. The Ectocarpus genus comprises in particular the species Ectocarpus siliculosus.

The term “PHLD.Pf” is intended to mean, without distinction, the gene encoding the PHLD phloroglucinol synthase of P. fluorescens, or the polypeptide encoded by this gene.

The term “PKS1.Es” or “PHLD.Es” is intended to mean, without distinction, the gene encoding the PKS1 phloroglucinol synthase of E. siliculosus, or the polypeptide encoded by this gene.

The term “PhlD” or “PHLD” denotes here a candidate gene encoding a candidate phloroglucinol synthase enzyme, or the polypeptide encoded by this gene. According to the nomenclature chosen by the inventors, the term “PhlD.ii” or “PHLD.ii” denotes here the candidate gene or the candidate polypeptide from a given organism. The letters “ii” represent the genus and the species to which said organism belongs.

The term “nucleic acid molecule” is intended to mean a polymer of any length of deoxyribonucleic acid (DNA), or polydeoxyribonucleotides, including in particular complementary DNAs or cDNAs, genomic DNAs, plasmids, vectors, viral genomes, isolated DNA, probes, primers and any mixture thereof; or a polymer of any length of ribonucleic acid (RNA), or polyribonucleotides, including in particular messenger RNAs or mRNAs, antisense RNAs; or mixed polyribo-polydeoxyribonucleotides. They encompass single-stranded or double-stranded, linear or circular, and natural or synthetic polynucleotides. In addition, a polynucleotide can comprise non-natural nucleotides and can be interrupted by non-nucleotide components. In the context of the present invention, the terms “nucleic acid”, “nucleic acid molecule”, “polynucleotide” and “nucleotide sequence” are used interchangeably.

The term “isolated molecule” is intended to mean a molecule, in particular a protein, a polypeptide, a peptide, a nucleic acid molecule, a plasmid vector, a viral vector or a host cell, which is extracted from its natural environment (that is to say separated from at least one other component with which it is naturally associated).

The term “polypeptide”, “protein” and “peptide” is intended to mean polymers of amino acid residues which comprise at least nine amino acids bonded via peptide bonds. The polymer may be linear, branched or cyclic. The polymer may comprise natural amino acids and/or amino acid analogues and it may be interrupted by non-amino acid residues. As a general indication and without however being bound thereto in the present application, if the amino acid polymer contains more than 50 amino acid residues, it is preferably referred to as a polypeptide or a protein, whereas if the polymer consists of 50 amino acids or less, it is preferably referred to as a “peptide”.

The term “vector” is intended to mean a carrier, preferably a nucleic acid molecule or a viral particle, which contains the elements required to enable one or more nucleic acid molecule(s) to be administered into, propagated in and/or expressed in a host cell or an organism.

From a functional point of view, this term encompasses maintenance vectors (cloning vectors), vectors for expression in various host cells or organisms (expression vectors), extrachromosomal vectors (for example multicopy plasmids) or integrating vectors (for example designed to integrate into the genome of a host cell and to produce additional copies of the nucleic acid molecule that it contains when the host cell replicates). This term also encompasses shuttle vectors (for example, which function both in prokaryotic hosts and/or eukaryotic hosts) and transfer vectors (for example for the transfer of nucleic acid molecule(s) into the genome of a host cell).

From a structural point of view, the vectors according to the invention may be natural, synthetic or artificial genetic sources, or a combination of natural and artificial genetic elements.

Thus, in the context of the invention, the term “vector” should be understood broadly while including plasmid vectors (or plasmids) and viral vectors.

A “plasmid” as used here denotes a replicatable DNA construct. Usually, plasmid vectors contain selectable marker genes which allow the host cells carrying the plasmid to be identified and/or selected positively or negatively in the presence of the compound corresponding to the selectable marker. A variety of positive and negative selectable marker genes are known in the art. By way of illustration, an antibiotic resistance gene can be used as a positive selectable marker gene for selecting a host cell in the presence of the corresponding antibiotic.

The term “viral vector” as used here refers to a nucleic acid vector which comprises at least one element of a viral genome and which can be packaged in a viral particle, or a viral particle. The viral vectors may be replication-competent or selective (for example, designed to replicate better or selectively in specific host cells), or may be genetically deactivated so as to be defective or deficient for replication.

The term “host cell” is intended to mean a cell containing a nucleic acid molecule according to the invention. Advantageously, the host cell is capable of expressing a polypeptide with phloroglucinol synthase activity and/or of producing the vector of the invention. Advantageously again, the host cell is capable of synthesizing phloroglucinol.

The host cell may consist of a single type of cells or of a group of different types of cells. The host cell may also be a hybrid cell, that is to say a cell resulting from the fusion of at least two cells of different type.

The host cell may belong to cultured cell lines, to primary cells, to stem cells or to proliferative cells. In the context of the invention, the term “host cells” comprises prokaryotic cells, lower eukaryotic cells such as yeast cells, and other eukaryotic cells such as insect cells, plant cells and mammalian cells (for example human or nonhuman cells, preferably nonhuman cells).

The term “host cell” comprises more broadly cells which contain or have contained the nucleic acid molecule according to the invention, and also the progeny of such cells. The host cell may for example be isolated or organized in a tissue or in an organ or else may be within a complete organism. In the case where the host cell is within a complete organism, said organism is not human.

It is thus clear that a “host cell” according to the present invention is a recombinant host cell, i.e. a cell housing an exogenous genetic material. Thus, a host cell is not a cell that exists naturally, but is a molecular biology tool obtained by genetic manipulation techniques.

The term “identity” is intended to mean an exact sequence correspondence between two polypeptides or two amino acid molecules. The “percentage identity” between two sequences depends on the number of identical residues common to the two sequences, and takes into account the number of intervals that must be introduced for an optimal alignment and the length of each interval. Various computer programs and mathematical algorithms are available in the prior art for determining the percentage identity between amino acid sequences, such as for example the Blast program available on the NCBI or ALIGN base (Atlas of Protein Sequence and Structure, Dayhoff (ed.), 1981, Suppl. 3 482-489). Programs for determining the homology between nucleotide sequences are also available in a specialized database (for example Genbank, the Wisconsin Sequence Analysis Package, the BESTFIT, FASTA and GAP programs). By way of illustration, the expression “at least 80% sequence identity”, as used here, represents 80%, 81%, 82%, 83%, 84%, 85%, 86%, 87%, 88%, 89%, 90%, 91%, 92%, 93%, 94%, 95%, 96%, 97%, 98%, 99% or 100%.

In the detailed description which follows, the embodiments may be taken alone or combined appropriately by those skilled in the art.

### Isolated Polypeptides and Use Thereof as Phloroglucinol Synthases

The focus herein is on the isolated polypeptides chosen from type III polyketide synthases of bacteria, in particular from type III polyketide synthases of gram+ bacteria, in particular from type III polyketide synthases of actinomycete bacteria.

In point of fact, the inventors have identified, entirely surprisingly, genes encoding new type III polyketide synthases in the genome of bacteria that was not known to encode this type of enzyme. The inventors have in particular demonstrated, most surprisingly, that these novel type III polyketide synthases have phloroglucinol synthase activity.

The present invention relates in particular to the use of isolated polypeptides chosen from type III polyketide synthases of bacteria as phloroglucinol synthases, preferably with the exclusion of the type III polyketide synthase of Pseudomonas fluorescens PHLD.Pf. The present invention relates in particular to the use of isolated polypeptides chosen from type III polyketide synthases of bacteria as phloroglucinol synthases, with the exclusion of the type III polyketide synthase of Pseudomonas fluorescens PHLD.Pf.

The present invention also relates in particular to the use of isolated polypeptides chosen from the type III polyketide synthases of gram+ bacteria, in particular from the type III polyketide synthases of actinomycete bacteria, as phloroglucinol synthases.

Advantageously, said polypeptide comprises at least one amino acid sequence having at least 50% identity with a sequence chosen from SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9 and SEQ ID No. 10.

Advantageously, said isolated polypeptide is chosen from the type III polyketide synthases of gram+ bacteria. Advantageously, said isolated polypeptide is chosen from the type III polyketide synthases of actinomycete bacteria chosen from the group consisting of Tsukamurella sp., Nocardia sp., Mycobacterium sp., and Gordonia sp., in particular Tsukamurella paurometabola, Tsukamurella tyrosinosolvens, Tsukamurella pseudospumae, Tsukamurella pulmonis, Tsukamurella sp. 1534, Nocardia farcinica, Mycobacterium marinum, Mycobacterium kansasii, Mycobacterium tuberculosis and Gordonia hydrophobica.

According to one embodiment, said polypeptide comprises at least one amino acid sequence preferably having at least 60% identity, more preferably at least 65% identity, more preferably at least 70% identity, even more preferably at least 75% identity, still more preferably at least 80% identity, even more preferentially at least 85% identity, even more preferentially at least 90% identity, more preferably at least 95% identity, more preferably at least 96% identity, even more preferably at least 97% identity, still more preferably at least 98% identity, and even more preferentially at least 99% identity, with a sequence chosen from SEQ ID No.: 1, SEQ ID No.: 2, SEQ ID No.: 3, SEQ ID No.: 4, SEQ ID No.: 5, SEQ ID No.: 6, SEQ ID No.: 7, SEQ ID No.: 8, SEQ ID No.: 9 and SEQ ID No.: 10.

According to one particularly advantageous embodiment, said polypeptide comprises at least one amino acid sequence chosen from SEQ ID No.: 1 SEQ ID No.: 2, SEQ ID No.: 3, SEQ ID No.: 4, SEQ ID No.: 5, SEQ ID No.: 6, SEQ ID No.: 7, SEQ ID No.: 8, SEQ ID No.: 9 and SEQ ID No.: 10.

In one preferred embodiment, the isolated polypeptide with phloroglucinol synthase activity has an amino acid sequence chosen from SEQ ID No.: 1 SEQ ID No.: 2, SEQ ID No.: 3, SEQ ID No.: 4, SEQ ID No.: 5, SEQ ID No.: 6, SEQ ID No.: 7, SEQ ID No.: 8, SEQ ID No.: 9 and SEQ ID No.: 10.

Advantageously, the isolated polypeptide with phloroglucinol synthase activity is chosen from the isolated polypeptide with phloroglucinol synthase activity PHLD.Tp of Tsukamurella paurometabola, the isolated polypeptide with phloroglucinol synthase activity PHLD.Tt of Tsukamurella tyrosinosolvens, the isolated polypeptide with phloroglucinol synthase activity PHLD.Tps of Tsukamurella pseudospumae, the isolated polypeptide with phloroglucinol synthase activity PHLD.Tpu of Tsukamurella pulmonis, the isolated polypeptide with phloroglucinol synthase activity PHLD.Tp of Tsukamurella sp. 1534, the isolated polypeptide with phloroglucinol synthase activity PHLD.Nf of Nocardia farcinica, the isolated polypeptide with phloroglucinol synthase activity PHLD.Mma of Mycobacterium marinum, the isolated polypeptide with phloroglucinol synthase activity PHLD.Mk of Mycobacterium kansasii, the isolated polypeptide with phloroglucinol synthase activity PHLD.Mt of Mycobacterium tuberculosis and the isolated polypeptide with phloroglucinol synthase activity PHLD.Gh of Gordonia hydrophobica.

### Isolated Nucleic Acid Molecules

The present invention relates to isolated nucleic acid molecules encoding at least one polypeptide chosen from type III polyketide synthases from bacteria, in particular type III polyketide synthases from actinomycete bacteria.

Advantageously, said polypeptide is as defined above.

According to one embodiment, the isolated nucleic acid molecule comprises a promoter controlling the expression of at least one nucleic acid sequence encoding a polypeptide as defined above. Thus, according to one embodiment, the present invention relates to an isolated nucleic acid molecule comprising at least one nucleic acid sequence encoding a polypeptide chosen from the type III polyketide synthases as defined above and also comprising a promoter controlling the expression of said at least one nucleic acid sequence.

Advantageously, the promoter is an exogenous promoter, in particular a yeast promoter, preferably a promoter chosen from ADH2 (pADH2) and CCW12 (pCCW12), more preferably a promoter chosen from ADH2 (pADH2) of Saccharomyces cerevisiae and CCW12 of S. cerevisiae, more preferably a promoter chosen from ADH2 of SEQ ID No.: 13 and CCW12 of SEQ ID No.: 14.

According to one embodiment, the isolated nucleic acid molecule comprises a transcription terminator for at least one nucleic acid sequence encoding a polypeptide as defined above. Thus, according to one embodiment, the present invention relates to an isolated nucleic acid molecule comprising at least one nucleic acid sequence encoding a polypeptide chosen from the type III polyketide synthases as defined above and also comprising a terminator controlling the expression of said at least one nucleic acid sequence.

Advantageously, the terminator is an exogenous terminator, in particular a yeast terminator, preferably the RPL3 terminator (tRPL3), more preferably the RPL3 terminator of S. cerevisiae, more preferably the RPL3 terminator of SEQ ID No.: 15.

According to one preferred embodiment, the isolated nucleic acid molecule comprises both a promoter and a terminator which are as defined above. Thus, according to one embodiment, the present invention relates to an isolated nucleic acid molecule comprising at least one nucleic acid sequence encoding a polypeptide chosen from the type III polyketide synthases as defined above and also comprising a promoter and a terminator controlling the expression of said at least one nucleic acid sequence.

According to one embodiment, the nucleic acid molecule also comprises an export sequence. Advantageously, this export sequence allows the secretion or excretion of the polypeptide(s) encoded by said nucleic acid molecule, in the cell medium.

According to one embodiment, the nucleic acid molecule is isolated from homologous strains in culture, preferably chosen from Tsukamurella sp., Nocardia sp., and Mycobacterium sp., in particular Tsukamurella pourometabola, Tsukamurella tyrosinosolvens, Tsukamurella pseudospumae, Tsukamurella pulmonis, Tsukamurella sp. 1534, Nocardia farcinica, Mycobacterium marinum, Mycobakterium kansasii, Mycobacterium tuberculosis and Gordonia hydrophobica.

According to one embodiment, the nucleic acid molecule is isolated from a heterologous vector or host cell comprising said molecule, said vector or said host cell being as defined above and as described below in the sections “Host cells” or “Vectors”.

According to one embodiment, the isolated nucleic acid molecule is synthesized in vitro by means of nucleic synthesis techniques that those skilled in the art know perfectly well how to define.

According to one embodiment, the isolated nucleic acid molecule is recombinant.

### Vectors

The present invention relates to vectors comprising at least one nucleic acid molecule as defined above.

Advantageously, the vector is a plasmid.

The vectors that are suitable in the context of the present invention comprise, without limitation, bacteriophage, plasmid or cosmid vectors for expression in prokaryotic host cells such as bacteria (for example E. coli, or bacteria of the Pseudomonas genus); vectors for expression in yeast (for example Saccharomyces cerevisiae, Schyzosaccharomyces pombe, Pichia pastoris); baculovirus vectors for expression in insect cell systems (for example, Sf 9 cells); viral and plasmid vectors for expression in plant cell systems (for example, the Ti plasmid, the cauliflower mosaic virus CaMV, the tobacco mosaic virus TMV); and also viral and plasmid vectors for expression in higher eukaryotic cells or organisms.

These vectors are generally commercially available (for example, from suppliers such as Invitrogen, Stratagene, Amersham Biosciences, Promega, etc.), available from deposit institutions such as the American Type Culture Collection (ATCC, Rockville, Md.), or have been the subject of numerous publications describing their sequence, their structures and the methods for producing them, so that those skilled in the art can apply them without difficulty.

Representative examples of suitable plasmid vectors comprise, without limitation, pREP4, pCEP4 (Invitrogen), pCI (Promega), pVAX (Invitrogen) and pgWiz (Gene Therapy System Inc).

### Host Cells

In another aspect, the present invention relates to host cells comprising at least one nucleic acid molecule or at least one vector as defined above.

According to various embodiments, said host cell, in particular said heterologous host cell mentioned above, can be a prokaryotic cell, a lower eukaryotic cell such as a yeast cell, and other eukaryotic cells such as insect cells, plant cells and mammalian cells (for example human or nonhuman cells, preferably nonhuman cells).

Advantageously, the host cell is a microorganism selected from bacteria, yeasts, fungi, algae and cyanobacteria.

The host cell is preferably a yeast, said yeast being in particular selected from the Saccharomyces, Candida, Ashbya, Dekkera, Pichia (Hansenula), Debaryomyces, Clavispora, Lodderomyces, Yarrowia, Zigosaccharomyces, Schizosaccharomyces, Torulaspora, Kluyveromyces, Brettanomycces, Cryptococcus and Malassezia genera.

Even more particularly, the yeast is selected from the species Saccharomyces cerevisiae, Saccharomyces boulardii, Saccharomyces douglasii, Saccharomyces bayanus, Zigosaccharomyces bailii, Schizosaccharomyces pombe, Dekkera brucelensis, Dekkera intermedia, Brettanomycces custersii, Brettanomycces intermedius, Kluyveromyces themotolerens, Torulaspora globosa and Torulaspora glabrata.

Even more particularly, the yeast is of the Saccharomyces genus, preferably of the species Saccharomyces cerevisiae.

According to one embodiment, the host cell comprises at least one copy of the nucleic acid molecule as defined above, integrated into its genome.

According to one embodiment, the host cell comprises a single copy of the nucleic acid molecule as defined above, integrated into its genome.

When the host cell is a yeast cell, the copy or copies of the nucleic acid molecule can be integrated at various loci, preferentially at the URA3 locus, at the JLP1 locus, at the LEU2 locus, or at the TRP1 locus of the genome of said yeast cell. When the host cell is a yeast cell and several copies of the nucleic acid molecule are integrated, the various copies can be integrated at the same locus, or else at different loci, preferentially at any one of the combinations of the URA3, JLP1, LEU2 and/or TRP1 loci.

Advantageously, the codons used in the nucleic acid molecule have been adapted for optimal expression in the host cell selected.

An optimal expression can in particular be obtained when the codons chosen are those preferentially used by the organism of origin of the host cell. The preferentially used codons are known for most organisms commonly used in the field. Those skilled in the art will be able to easily determine the most advantageous codons to be used as a function of the host cell chosen.

To this effect, those skilled in the art know which technique to use in order to modify the codons of the nucleic acid molecule. The codons can for example be modified by in vitro site-directed mutagenesis using a sample of the nucleic acid molecule of which the codons are to be adapted, by means of an amplification by polymerase chain reaction (PCR). Alternatively, the nucleic acid molecule can be synthesized in vitro directly with the optimized codons.

The host cells can be cultured in small-scale and large-scale, aerobic or anaerobic bioreactors, in flasks or in Petri dishes. The culture can be performed at a temperature, at a pH, in a culture medium and at an oxygen content that are suitable for a given host cell.

### Methods

Method for Producing a Polypeptide with Phloroglucinol Synthase Activity

The present invention also relates to a method for producing a polypeptide with phloroglucinol synthase activity as defined above.

According to one embodiment, the method for producing a polypeptide with phloroglucinol synthase activity as defined above comprises at least the steps consisting in:
(i) introducing a nucleic acid molecule or a vector as described above into a suitable host cell in accordance with the preceding description; and
(ii) culturing, in vitro, said host cell obtained in step (i) under conditions which allow the growth of said host cell and/or the expression of said nucleic acid molecule, so as to produce said polypeptide.

According to another embodiment, the method for producing a polypeptide with phloroglucinol synthase activity as defined above comprises at least the step consisting in:
(i) culturing, in vitro, a host cell expressing said polypeptide, for example a host cell as described above, under conditions which allow the growth of said host cell and/or the expression of the nucleic acid molecule contained in said host cell, so as to produce said polypeptide.

According to one possible embodiment, the method for producing a polypeptide with phloroglucinol synthase activity comprises at least one additional step chosen from the steps consisting in:
(α) recovering the cells expressing said polypeptide, obtained after the culturing step; and
(β) purifying the polypeptide from the cells recovered in step (α).

### Method for Producing Phloroglucinol

The present invention also relates to a method for producing phloroglucinol.

According to one embodiment M1, the method for producing phloroglucinol comprises at least the steps consisting in:
(i) obtaining cells by implementing one of the methods described above;
(ii) bringing the cells obtained in step (i) into contact with a suitable substrate;
(iii) incubating the mixture resulting from step (ii) under conditions suitable for producing phloroglucinol;
(iv) optionally, recovering the reaction medium comprising the phloroglucinol, obtained after step (iii); and
(v) optionally, purifying the phloroglucinol from the reaction medium of step (iv).

According to another embodiment M2, the method for producing phloroglucinol comprises the steps consisting in:
(i) bringing a host cell expressing the polypeptide with phloroglucinol synthase activity as defined above, for example a host cell as defined above, into contact with a suitable substrate;
(ii) culturing, in vitro, the host cell of step (i) under conditions which allow the growth of said host cell and/or the expression of the nucleic acid molecule contained in said host cell, so as to produce phloroglucinol;
(iii) optionally, recovering the culture medium comprising the phloroglucinol, obtained after step (ii); and
(iv) optionally, purifying the phloroglucinol from the culture medium of step (iii).

For the purposes of methods M1 and M2, the substrate is a carbon source. Advantageously, the carbon source is a pure carbon source or an industrial coproduct (such as molasses or green syrup, for example from the sugar industry). Preferably, the substrate in the pure carbon source or the industrial coproduct is a simple sugar, such as glucose (or dextrose), fructose, galactose, mannose, sucrose, lactose or maltose; a complex sugar, such as a monosaccharide, a disaccharide or trisaccharides, or else a polysaccharide such as starch; an alcohol, such as ethanol; an acid; a fatty acid and the ester derivative thereof; or a mixture of sugars, of alcohols, of acids and/or of fatty acids or the ester derivatives thereof.

Preferably, the substrate is glucose. Alternatively, the substrate is ethanol.

According to another embodiment M3, the method for producing phloroglucinol comprises at least the steps consisting in:
(i) bringing at least one polypeptide obtained in step (ß) of the method as described above into contact with a suitable substrate;
(ii) incubating the mixture resulting from step (i) under conditions suitable for producing phloroglucinol;
(iii) optionally, recovering the reaction medium comprising the phloroglucinol, obtained after step (ii); and
(iv) optionally, purifying the phloroglucinol from the reaction medium of step (iii).

According to another embodiment M4, the method for producing phloroglucinol comprises at least the steps consisting in:
(i) bringing at least one polypeptide as defined above into contact with a suitable substrate;
(ii) incubating the mixture resulting from step (i) under suitable conditions for producing phloroglucinol;
(iii) optionally, recovering the reaction medium comprising the phloroglucinol, obtained after step (ii); and
(iv) optionally, purifying the phloroglucinol from the reaction medium of step (iii).

For the purposes of these methods M3 and M4, the substrate is a thioester. Advantageously, the substrate is an acyl-Coenzyme A (or acyl-CoA) such as malonyl-CoA, acetyl-CoA, hexanoyl-CoA, decanoyl-CoA, lauroyl-CoA and palmitoyl-CoA, or a mixture thereof. Preferably, the substrate is malonyl-CoA.

According to one preferred embodiment, the purification of the phloroglucinol is carried out by liquid-liquid extraction.

The examples which follow aim to illustrate the present invention without any limitation. The enzymes respectively encoded by the PHLD gene of Pseudomonas fluorescens (Zha et al., 2006), and by the PKS1 gene of Ectocarpus siliculosus (Meslet-Cladière et al., 2013) are used therein as controls.

### EXAMPLES

### Example 1: Identification of New Candidate Phloroglucinol Synthases

Up until the present invention, only two phloroglucinol synthases had been identified and characterized:
the enzyme encoded by the PHLD gene of Pseudomonas fluorescens (Zha et al., 2006), and
the enzyme encoded by the PKS1 gene of Ectocarpus siliculosus (Meslet-Cladière et al., 2013).

The inventors have now discovered and characterized new phloroglucinol synthases using genetic and functional analyses.

As indicated in the introduction above, the exact functional characterization of a polyketide synthase is complex since this class of enzymes groups together proteins having high sequence similarities although they can catalyse substantially dissimilar reactions and recognize entirely different substrates.

### 1.1. Selection of New Candidate Enzymes

In order to identify new phloroglucinol synthases, the inventors identified sequences encoding putative type III polyketide synthases. The sequences of these putative type III polyketide synthases thus identified by the inventors were analysed and aligned with respect to one another using in particular as a basis the type III polyketide synthase alignment published by Meslet-Cladière et al. 2013.

The analysis of this sequence alignment resulted in the selection of a group of candidate enzymes, the protein sequences of which are close to that of the product of the PKS1.Es gene of the alga Ectocarpus siliculosus (Tables 1 and 2).

TABLE 1
Putative type III polyketide synthases
identified. The shaded rows show the enzymes
which have a known phloroglucinol synthase activity.

| | | Known | | |
|---|---|---|---|---|
| | Reference in | or putative | | |
| Name | the databases | function | Species | Kingdom |
| PHLD. | AAY95147.1 | PhlD | Pseudomonas | Gammaproteo- |
| Pf | | type III | fluorescens | bacteria/ |
| | | polyketide | Pf-5 | pseudomonals |
| | | synthase | | (bacteria) |
| PKS1. | CBN76919.1 | III | Ectocarpus | Ochrophytes |
| Es | | polyketide | siliculosus | (algae) |
| | | synthase | | |
| | | without | | |
| | | signal | | |
| | | peptide | | |
| | | 2-32 | | |
| PHLD. | WP_ | polyketide | Tsukamurella | Actinobacteria/ |
| Tp | 013126955.1 | synthase | pauro- | tsukamurella |
| | | | metabola | (bacteria) |
| PHLD. | WP_ | polyketide | Tsukamurella | Actinobacteria/ |
| Tt | 068525790.1 | synthase | tyrosino- | tsukamurella |
| | | | solvens | (bacteria) |
| PHLD. | WP_ | polyketide | Tsukamurella | Actinobacteria/ |
| Tps | 068569959.1 | synthase | pseudo- | tsukamurella |
| | | | spumae | (bacteria) |
| PHLD. | WP_ | polyketide | Tsukamurella | Actinobacteria/ |
| Tpu | 068567598.1 | synthase | pulmonis | tsukamurella |
| | | | | (bacteria) |
| PHLD. | WP_ | polyketide | Tsukamurella | Actinobacteria/ |
| Tsp | 019202763.1 | synthase | sp. 1534 | tsukamurella |
| | | | | (bacteria) |
| PHLD. | WP_ | polyketide | Nocardia | Actinobacteria/ |
| Nf | 011211464.1\| | synthase | farcinica | corynebacteria |
| | | | | (bacteria) |
| PHLD. | WP_ | polyketide | Myco- | Actinobacteria/ |
| Mma | 012393954.1\| | synthase | bacterium | corynebacteria |
| | | | marinum | (bacteria) |
| PHLD. | ZP_ | polyketide | Myco- | Actinobacteria/ |
| Mk | 04749411.1\| | synthase | bacterium | corynebacteria |
| | | | kansasii | (bacteria) |
| | | | ATCC 12478 | |
| PHLD. | WP_ | polyketide | Myco- | Actinobacteria/ |
| Mt | 031677738.1 | synthase | bacterium | corynebacteria |
| | | | tuberculosis | (bacteria) |
| PHLD. | WP_ | polyketide | Gordonia | Actinobacteria/ |
| Gh | 066172590.1 | synthase | hydrophobica | actinomycetales |
| | | | | (bacteria) |

TABLE 2
Protein sequences of the putative type III polyketide synthases identified

| SEQ ID No.: | Name | Protein sequence |
|---|---|---|
| SEQ ID NO: 1 | PHLD.Tp | MIAPQIRLGEPDTTPLPDPLWHGAPPAPPTTVAVIESLATGSPAQAHGQSVSAERV |
| | | AARFADPIQAERIRRVYANTAVATRHLAIDPLSDDFADFSARPDTIRQRMDLYFEHA |
| | | APLAIETARRALGAVDATEVGQLIFVTSTGFLAPGVDVAVTRSLGLPASTSRVVINFM |
| | | GCAAAMNALRVASDFVRAHPDRKSLLVCLELSSVNAVFAGDPNDVVISSLFADGCG |
| | | AALIGASEVGHPLPAGHIVVRDTFAHLLDDAEDGIVLGVNANGITCELAHSLPRYILD |
| | | GVSPVIDGVLARNGLGREDVAHWAIHPGGPKIIESASAALGLPRSASQTSWAVLAE |
| | | HGNMLSVSLLFVLERLLGARAAGGTQETGLAFSFAPGVTVEGFLFDVVGGPS |
| SEQ ID NO: 2 | PHLD.Tt | MTMNAGSQGAPALDVPAPVLPAPGTWLGAPPAPPTSVAVIESLATGSPAQTYGQ |
| | | AESADRVAARFDDPRQAERIRRVYAKTRVDERHLAIDPLTPEFAEFSTRPDTVRERM |
| | | DLFYEHAAPLAVDTARRALGVGTEHEFDPADVGQLVFVTSTGFLAPGVDVAVIRAL |
| | | GLAPQTSRVVINFMGCAAAMNALRVSTDYVRAHPDRKSLMICLELSSVNAVFSGDP |
| | | NDVVISSLFADGCGAAVIGASEVGHPLPGGRIVVRDTFTHLLDGAEDGIVLGVNANG |
| | | ITCELAESLPQYIVDGVAPVIDEVLGRNDLGREAVAHWAIHPGGPKIIESAATALGLP |
| | | DEASRTSWDVLAEHGNMLSVSLLFVLERLLAQVADGAATQPDGAPTTGMAFSFAP |
| | | GVTVEGFLFDVVTGDQPGE |
| SEQ ID NO: 3 | PHLD.Tps | MAAPELPGTSVWRGAPPAPPTSVAVIESLATGSPSQAYDQAESADRVASRFDDPR |
| | | QAERIRRVYAKTRVAERHLAIDPLTPEFAAFSTRPDTIRERMDLFFEHAAPLAIDTARR |
| | | ALGTVDPADVGQLVFVTSTGFLAPGVDVAVIRALGLSPGTSRVVINFMGCAAAMN |
| | | ALRVSTDYVRAHPDRKSLMICLELSSVNAVFSGDPNDVVISSLFADGCGAALIGASEV |
| | | GHPLPAGNIVVRDTFSHLLDGAEDGIVLGVNADGITCELAESLPQYIVDGVAPVVDG |
| | | VLRRNGLDRDAVAHWAIHPGGPKIIESASTALGLPDEASRLSWDVLAGHGNMLSVS |
| | | LLFVLERLLAQVASDGADGAPSTGMAFSFAPGVTVEGFLFDVVTTGE |
| SEQ ID NO: 4 | PHLD.Tpu | MNTTEQQSVLPQQAWLGAPPAPPTSVAVIESLATSSPTQTYGQAESADRVAARFD |
| | | DPRQAERIRRVYAKTRVTERHLAIDPLTPEFAEFSARPDTVRERMDLFFEHAAPLAIE |
| | | TARRALGDNAATDIGQLVFVTSTGFLAPGVDVAVIRALGLAPQTSRVVINFMGCAA |
| | | AMNALRVSTDYVRAQPDRKSLMICLELSSVNAVFSGDPNDVVISSLFADGCGAAVI |
| | | GASEVGHPLPSGQIVVRDTFTHLLDGAEDGIVLGVNANGITCELAESLPQYIVNGVA |
| | | PVVDAVLDRNGLGREAVAHWAIHPGGPKIIESAAAALGLPDDASRLSWDVLAEHG |
| | | NMLSVSLLFVLERLRAQVASDGADGAPSTGMAFSFAPGVTVEGFLFDVVTTGE |
| SEQ ID NO: 5 | PHLD.Tsp | MNIQDRSTVAPDSPALGFPPAPPTSVAVIESLATGSPSGVHAQAESADRVASRFDD |
| | | PAQAERIRRVYTKTRVARRHLAIDPLDEDFAAFSARPDTIRERMDLFAEHASPLAVDT |
| | | ARRALGAVDPADVGQLVFVTSTGFLAPGVDVAIVRALGLPATTSRVIVNFMGCAAA |
| | | MNALRVASDFVRAHPDRKSLMVCLELSSVNAVFSGDPNDVVISSLFADGCGAAVIG |
| | | ASEVGHPLPGGSIVVRDTFAHLLDGAEDGIVLGVNANGITCELAESLPRYIVDGVRPV |
| | | IDGVLARNGLGHADVAHWAIHPGGPKIIESASAALGLPAAASATSWQVLAEHGNM |
| | | LSVSLLFVLERMLSGLPDGAPSTGMAFSFAPGVTVEGFLFDIVRDGAPRPGALGGRP |
| | | A |
| SEQ ID NO: 6 | PHLD.Nf | MSITVDEGGARPATEPRQRIHPDLGHAHTPMPPAPPVTIGVVEGIATGSPAQIVDQ |
| | | AEAAERVAALFTDPAQRARIARVYEKTRIETRRMAVDPTAPEFRSFSRQPGTLRERM |
| | | NLFYRHAAPLAVDVAGRALADSGAAAADIGLLVFVTSTGFIAPGVDVAVLRELGLVP |
| | | TVGRVVVNFMGCAAAMNGIRTGVDYVRAHPDKKALVVCLELSSVNAVFADDVND |
| | | VIIHSLFGDGCGAVVLGASQARQPLAPGRVVVRDSFSHLFDAAEDGIVLGVDHNGIT |
| | | CELSENLPRYIYRGVDPVVREVLARNGLRKSDIDLWAIHPGGPKIIEESVRSLGLSPEQ |
| | | AAPSWDVLARHGNMLSVSLIFVLEQMIAQSATAEPISTGVAFSFAPGVTLEGLIFDII |
| | | RR |
| SEQ ID NO: 7 | PHLD.Mma | MSTAAEGGAIRRAGHEPRYDLAQLPPAPPTTVAVIEGMATGAPQRVVAQADAAA |
| | | RVSELFVDPQQRERISRIYDKTRIDTRRMAVDPLDDEFDEFRREPATIRDRMNLFYQ |
| | | HAVPLAVDVAARALDGLPYAPDEIGQLVFVTSTGFIAPGVDVEIVKQLGLPRSISRVV |
| | | VNFMGCAAAMNAIRTATNYVRAHPSMKALVVCIELSSVNAVFADDINDVVIHSLFG |
| | | DGCGALVIGASQVQQPLPAGNVVIRSSFSQLLDDSEDGIVLGVNHDGITCELSENLP |
| | | SYIYRSVDPVVAEMLRDNGLSKADIDLWAIHPGGPKIIEQSARSLGIPVGRAVQSWD |
| | | VLAQFGNMLSVSLIFVLEMMVAQAESDKPISTGVAFAFAPGVTVEGMLFDIIRR |
| SEQ ID NO: 8 | PHLD.Mk | MSSAADGGAPVADVPGYEPHYDLAQLPPAPPTTVAVIEGMATGVPQRVVRQSDA |
| | | AARVAQMFVDPQQRERVSRVYAKTRIDTRRMAVNPLDAEFDAFRREPATIRDRMS |
| | | LFYRHAVPLAVEVTRRALAGLSYGADEIGLLVFVTSTGFVAPGVDVAIVKELGLSRAIS |
| | | RVVVNFMGCAAAMNAIRTATNYVRAHPAMKALVVCIELCSVNAVFADNVKDVVI |
| | | HSLFGDGCAALVIGASQVQQQLPAGSVVIRSNFSQLLDDAEDGIVLGVNHDGITCEL |
| | | SENLPDYIYRGVAPVVANVLYDNGLQQSDIDLWAIHPGGPKIIEQSVRSLGIGVECAA |
| | | PSWDVLARYGNMLSVSLIFVLEMMVQQAESEKPLSTGVAFAFAPGVTVEGMLFDI |
| | | VRR |
| SEQ ID NO: 9 | PHLD.Mt | MNVSAESGAPRRAGQRHEVGLAQLPPAPPTTVAVIEGLATGTPRRVVNQSDAADR |
| | | VAELFLDPGQRERIPRVYQKSRITTRRMAVDPLDAKFDVFRREPATIRDRMHLFYEH |
| | | AVPLAVDVSKRALAGLPYRAAEIGLLVLATSTGFIAPGVDVAIVKELGLSPSFGDGCA |
| | | ALVIGASQVQEKLEPGKVVVRSSFSQLLDNTEDGIVLGVNHNGITCELSENLPGYIFS |
| | | GVAPVVTEMLWDNGLQISDIDLWAIHPGGPKIIEQSVRSLGISAELAAQSWDVLAR |
| | | FGNMLSVSLIFVLETMVQQAESAKAISTGVAFAFGPGVTVEGMLFDIIRR |
| SEQ ID NO: 10 | PHLD.Gh | MPAPVTTVAVIEAVATGAPATVHPQTRAAEQVAELYDDPALQERIRRLYRNTRVQT |
| | | RHLAVDPMTPEFQEFSSRPATVRTRMNDYFHHAVPLAVDVARRALAGVTDPATEI |
| | | GQIIFVTSTGFIAPGVDVAVITELGLAPTVHRVIINFMGCAAAINGISTATDHVRANP |
| | | DSRALLICLELSSVNAVFGADPVELVTHSLFGDGCGAMLIGASPVGRRLAPGQLVVR |
| | | DTFSHLFHDTGDGIVLGVNDDGITCELAQELPSYIRRGVGPAIDAALNRSRLRRDDIA |
| | | HWAIHPGGPAIIEQSVAALDLPPDRAATSWEVLAEYGNMLSVSLVFVLEKLIAAGAH |
| | | GRGQAPETGVAFSFAPGVALEGMLFDLVC |

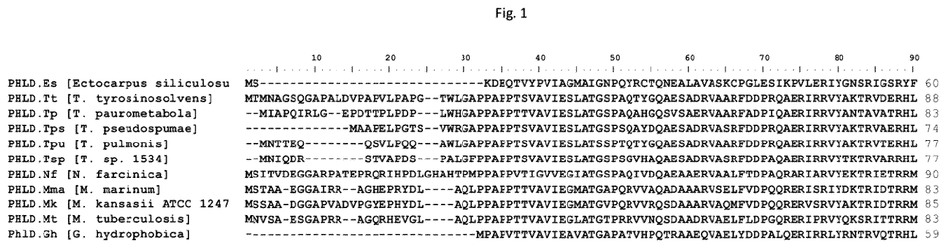

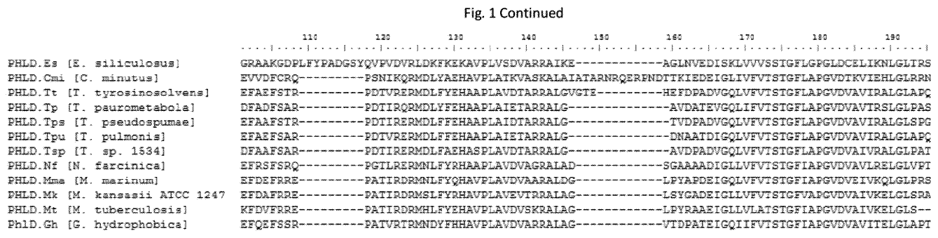

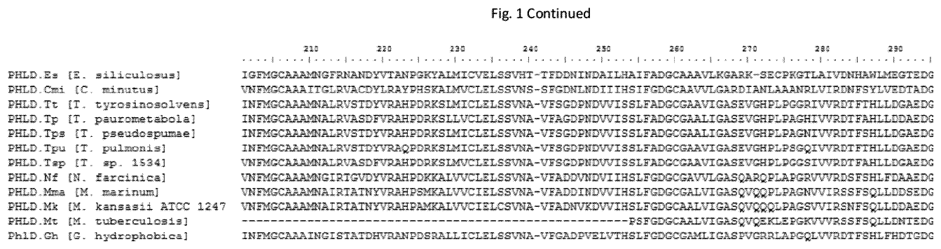

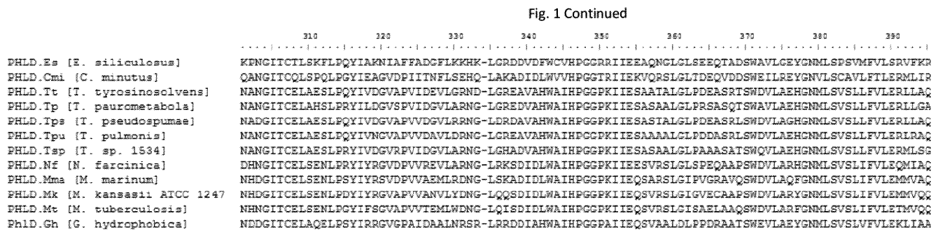

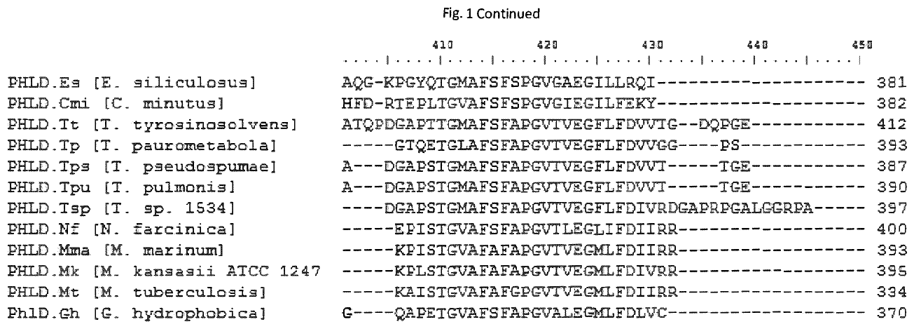

FIG. 1 presents the alignment of the protein sequences of the candidate enzymes listed in Table 1. The alignment of the ten candidate enzymes selected with the PKS1.Es enzyme was carried out using the Clustal W software.

Table 3 presents the matrix of the sequence identities that exist between these various candidate enzymes and PHLD.Pf and PKS1.Es.

TABLE 3
Matrix of the sequence identities that exist between the various candidate enzymes.

| | PHLD. | PHLD. | PHLD. | PHLD. | PHLD. | PHLD. | PHLD. | PHLD. | PHLD. | PHLD. | PHLD. | PKS1. |
|---|---|---|---|---|---|---|---|---|---|---|---|---|
| | Tt | Tp | Tps | Tpu | Tsp | Nf | Mma | Mk | Mt | Gh | Pf | Es |
| PHLD.Tt | ID | 74.7 | 84.2 | 85.4 | 75.7 | 58.2 | 55.8 | 55.3 | 44.6 | 55.8 | 19.3 | 36.3 |
| PHLD.Tp | 74.7 | ID | 76.3 | 76 | 76.5 | 55.4 | 55 | 54.7 | 42.7 | 58.4 | 21.3 | 34 |
| PHLD.Tps | 84.2 | 76.3 | ID | 87.2 | 77.9 | 57.6 | 57.7 | 56.3 | 45.2 | 58.3 | 20.4 | 37.3 |
| PHLD.Tpu | 85.4 | 76 | 87.2 | ID | 76.8 | 57.8 | 56.2 | 54.7 | 44.7 | 58.9 | 20.3 | 37 |
| PHLD.Tsp | 75.7 | 76.5 | 77.9 | 76.8 | ID | 56 | 56.3 | 55 | 45.3 | 54.8 | 19.9 | 35 |
| PHLD.Nf | 58.2 | 55.4 | 57.6 | 57.8 | 56 | ID | 67.5 | 67.2 | 55.5 | 55.7 | 19.1 | 35.9 |
| PHLD.Mma | 55.8 | 55 | 57.7 | 56.2 | 56.3 | 67.5 | ID | 82.7 | 65.6 | 54.9 | 19.9 | 35.8 |
| PHLD.Mk | 55.3 | 54.7 | 56.3 | 54.7 | 55 | 67.2 | 82.7 | ID | 66 | 52.8 | 19 | 35.4 |
| PHLD.Mt | 44.6 | 42.7 | 45.2 | 44.7 | 45.3 | 55.5 | 65.6 | 66 | ID | 44.3 | 15.1 | 27.7 |
| PHLD.Gh | 55.8 | 58.4 | 58.3 | 58.9 | 54.8 | 55.7 | 54.9 | 52.8 | 44.3 | ID | 21.1 | 35.9 |
| PHLD.Pf | 19.3 | 21.3 | 20.4 | 20.3 | 19.9 | 19.1 | 19.9 | 19 | 15.1 | 21.1 | ID | 23.1 |
| PKS1.Es | 36.3 | 34 | 37.3 | 37 | 35 | 35.9 | 35.8 | 35.4 | 27.7 | 35.9 | 23.1 | ID |

The results show that the phylogenetic distance that exists between the candidate enzymes and the bacterial enzyme PHLD.Pf is considerable since less than 25% identity is observed between these two groups. It is not therefore possible to assign, a priori, a phloroglucinol synthase function to the putative polyketide synthases studied because of this strong sequence divergence.

### 1.2. Measurement of the Phloroglucinol Synthase Activities in Yeast

In order to identify the phloroglucinol synthases among the candidates identified above, a method of extracting and assaying phloroglucinol was developed as detailed below.

### 1.2.1. Phloroglucinol Extraction Method

The method was developed using resorcinol as internal standard. Various tests resulted in the development of a liquid-liquid extraction method carried out at pH 4.0 in the presence of ethyl acetate as solvent, and by saturating the aqueous phase with NaCl. The extraction is carried out for 30 min with circular shaking. The organic phase is removed and the ethyl acetate solvent is evaporated off under a stream of nitrogen N₂ at 30° C. The dry extract obtained after complete evaporation is then taken up in a predetermined volume of a 50%-50% ethanol/H₂O mixture.

The extraction yield was measured by mass spectrometry after high pressure chromatography on a C18 column (dimensions: 100 mm×2.1 mm; particle size: 1.7 μm) using a 0.03% methanoic acid (HCOOH)/acetonitrile (ACN) gradient.

The extraction yields were determined using solutions of phloroglucinol and resorcinol prepared in the culture medium used for the growth of the yeasts. The phloroglucinol concentrations correspond to the bottom (20 μg.ml⁻¹) and top (200 μg.ml⁻¹) points of the assay range. The resorcinol concentration corresponds to the concentration added as internal standard during the assays (200 μg.ml⁻¹). The results are presented in Table 4.

TABLE 4
Extraction yield for phloroglucinol (20 and 200 μg · ml⁻¹) and
for resorcinol (200 μg · ml⁻¹), extracted with ethyl acetate,
according to the method described

| Product | Phloroglucinol | | Resorcinol (EI) |
|---|---|---|---|
| Concentration of phloroglucinol or of | 20 | 200 | 200 |
| resorcinol in the culture medium | | | |
| (μg · ml⁻¹) | | | |
| YLD (%) | 76 | 89 | 82 |

### 1.2.2. Development of a UPLC/UV and UPLC/MS Analysis Method

A method of analysis by UPLC chromatography and UV (ultraviolet radiation) absorbance measurement was developed. The extract is chromatographed on a pentafluorophenyl propyl (PFP) column having the dimensions 100 mm×2.1 mm; particle size: 1.8 μm, according to a 0.1% HCOOH/ACN-0.1% HCOOH gradient. The phloroglucinol is detected by UV at 230 nm. A UPLC-mass spectrometry (UPLC/Mass) method was also developed.

The quantification is carried out using a range of 20 to 200 μg.ml⁻¹ of phloroglucinol diluted in yeast culture medium (Yeast Extract 1%, BactoPeptone 2%) in the presence of a fixed amount of resorcinol, used as internal standard. The amount of phloroglucinol is determined by calculating the surface ratios of the phloroglucinol/resorcinol chromatography peaks.

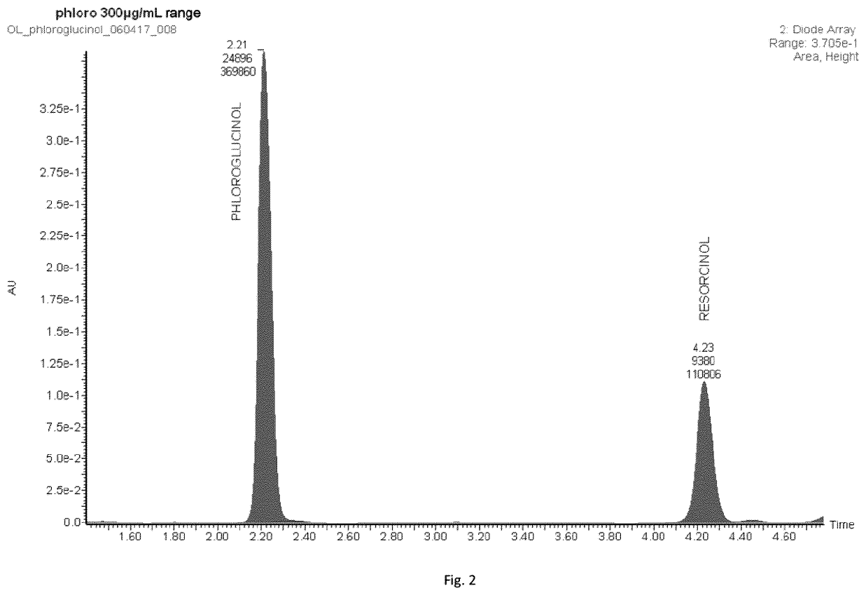

FIG. 2 presents an example of phloroglucinol/resorcinol chromatography peaks obtained on a PFP column.

This assay method thus makes it possible to reliably measure, qualitatively and quantitatively, the phloroglucinol present in a sample.

The UPLC-mass spectrometry (UPLC/mass) method makes it possible to assay the samples containing a phloroglucinol concentration ranging from 2 to 50 μg.ml⁻¹.

### Example 2: Identification of New Functional Phloroglucinol Synthases

2.1. Expression of the Candidate Genes in the Saccharomyces cerevisiae Yeast in Multicopy Form

The 10 candidate genes (PHLD.Tp, PHLD.Tt, PHLD.Tps, PHLD.Tpu, PHLD.Tsp, PHLD.Nf, PHLD.Mma, PHLD.Mk, PHLD.Mt and PHLD.Gh) selected and described above, and also the PHLD.Pf and PKS1.Es genes encoding the two phloroglucinol synthases identified to date, used as controls (see Tables 1 and 2), were synthesized by adapting the codons used for optimal expression in the S. cerevisiae yeast.

This codon adaptation was carried out in order to optimize the expression of these different genes in the yeast cells (these 12 synthetic genes encode proteins strictly identical to the proteins expressed by the organisms of origin). Each of these genes was placed under the control of the same yeast promoter ADH2 (pADH2) which allows their expression in particular when the culture medium contains ethanol as carbon source. The transcription terminator of the RPL3 yeast gene (tRPL3) was placed downstream of each of the 12 genes placed under the control of the ADH2 promoter.

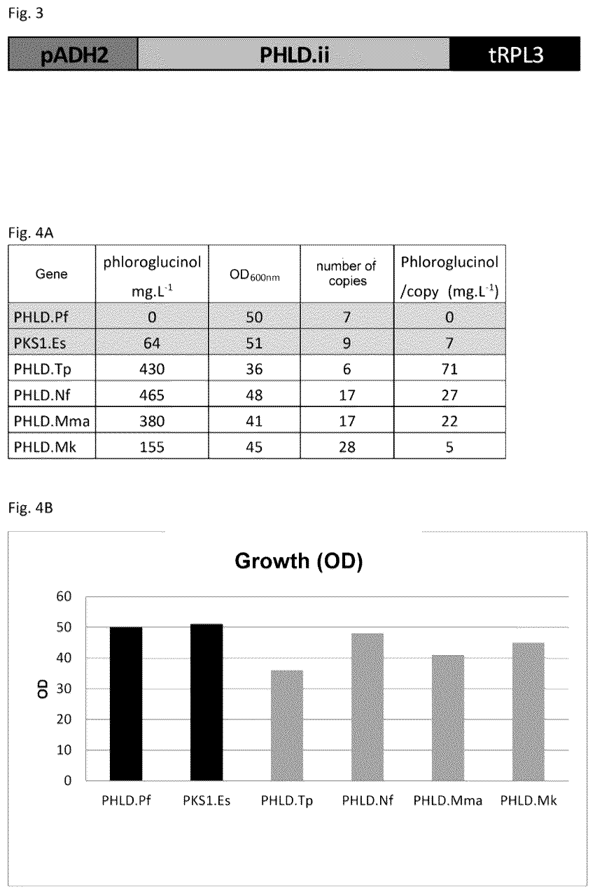

FIG. 3 shows an example of a gene unit thus constructed for a candidate or a given control (PHLD.ii).

The various gene units thus constructed were independently integrated at the URA3 locus of the genome of a wild-type strain of the S. cerevisiae yeast. The wild-type strain used is the commercial strain W303 (genotype: MAT-a, his3, leu2, trp1, ura3, ade−). The integration technique used allows the integration of a variable number of copies of each gene unit. For each construct, the number of copies of gene units integrated was determined by quantitative PCR according to the conventional Taqman method.

The yeast strains expressing various numbers of copies of each of the 10 PHLD.ii candidate genes or of the PHLD.Pf and PKS1.Es control genes described above were cultured in the presence of 20 g.l⁻¹ of ethanol as carbon source for 48 hours at 30° C. The 12 yeast strains independently obtained after transformation and integration, at the URA3 locus, of the gene units described above were thus analysed for their capacity to produce phloroglucinol. The W303 wild-type parental strain was cultured under the same conditions and used as a control.

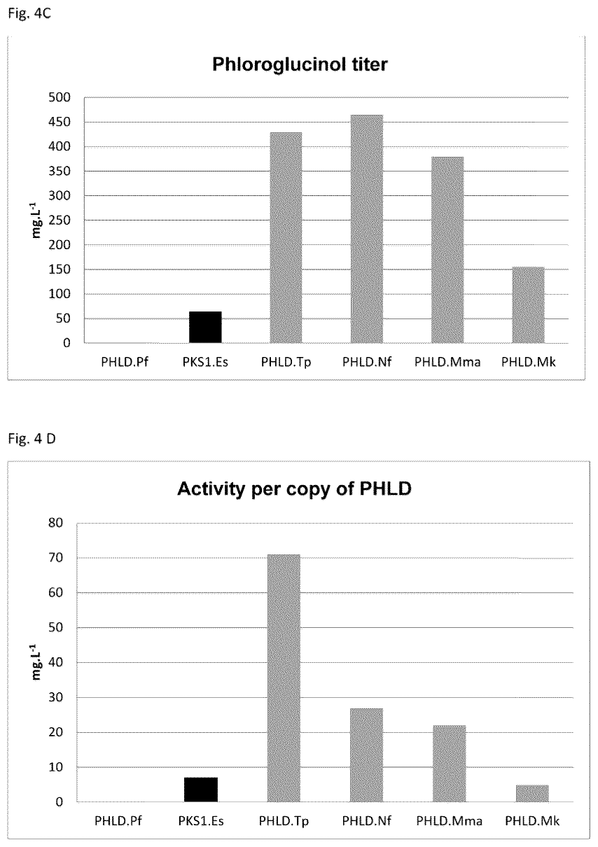

The optical densities (ODs) of the various cultures were measured at 600 nm (OD₆₀₀), thus indicating the level of growth of each strain (FIG. 4A and B).

The capacity of the various yeast strains expressing the various PHLD.ii genes under the control of the ADH2 promoter to synthesize phloroglucinol was tested using the extraction and assay method developed as described in section 1.2 above. The phloroglucinol production level (in mg.l⁻¹) was measured in the culture medium (FIGS. 4A and C).

FIG. 4 shows that all of the strains expressing different numbers of copies of the PHLD.Tp, PHLD.Tt, PHLD.Tps, PHLD.Tpu, PHLD.Tsp, PHLD.Nf, PHLD.Mma, PHLD.Mk, PHLD.Mt and PHLD.Gh genes produce a significant amount of phloroglucinol which is excreted into the culture medium (FIG. 4A, C and D). These results thus indicate that each of these 10 genes expresses a phloroglucinol synthase that is active in yeast.

Furthermore, as expected, no phloroglucinol production was measured in the W303 control parental strain (data not shown).

Thus, this functional study made it possible to identify ten new phloroglucinol synthases, encoded respectively by the PHLD.Tp, PHLD.Tt, PHLD.Tps, PHLD.Tpu, PHLD.Tsp, PHLD.Nf, PHLD.Mma, PHLD.Mk, PHLD.Mt and PHLD.Gh genes. This study reveals for the first time that Gram+ bacteria, in particular actinomycete bacteria encode functional phloroglucinol synthases (PHLD.Tp, PHLD.Tt, PHLD.Tps, PHLD.Tpu, PHLD.Tsp, PHLD.Nf, PHLD.Mma, PHLD.Mk, PHLD.Mt and PHLD.Gh).

This study also reveals for the first time that the PKS1.ES enzyme is functional when it is expressed in a heterologous eukaryotic system, namely yeast. This study also reveals, unexpectedly, that PHLD.Pf does not encode a phloroglucinol synthase that is functional in yeast. This result is surprising since the enzyme encoded by PHLD.Pf exhibits a phloroglucinol synthase activity demonstrated when it is expressed in Escherichia coli (Achkar et al., 2005).

2.2. Expression of the Candidate Genes in the Saccharomyces cerevisiae Yeast in the Form of a Single Copy

The production of phloroglucinol by strains having integrated only a single copy of the PHLD candidate gene selected and identified as being functional in yeast (see the results of the preceding section and FIG. 4) was also evaluated. The PKS1.Es gene encoding phloroglucinol synthase that is functional in yeast according to the results described in section 2.1 above was used as positive control. The PHLD.Pf gene encoding phloroglucinol synthase that is not functional in yeast according to the results described in section 2.1 above was used as negative control.

Each of these genes was placed either under the control of the yeast promoter ADH2 (pADH2), which allows their expression in particular when the culture medium contains ethanol as carbon source, or under the control of the yeast promoter CCW12 (pCCW12), which allows their expression, in particular during glycolysis, when the culture medium contains glucose as carbon source. The transcription terminator of the RPL3 yeast gene (tRPL3) was placed downstream of each of the constructs.

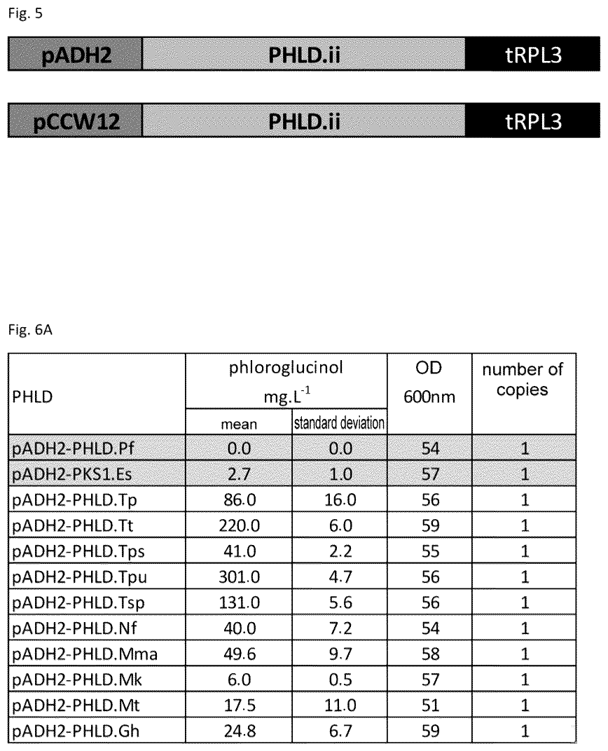

The details of the various gene constructs produced is reported in FIG. 5.

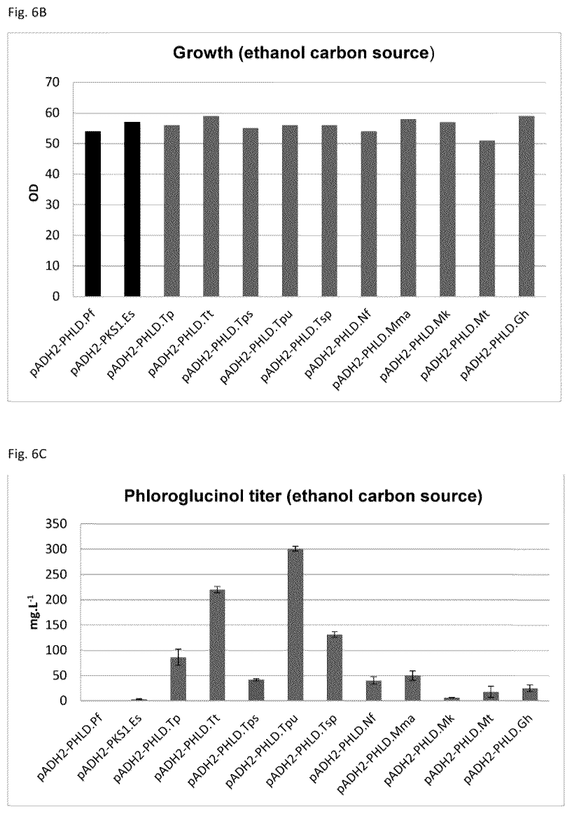

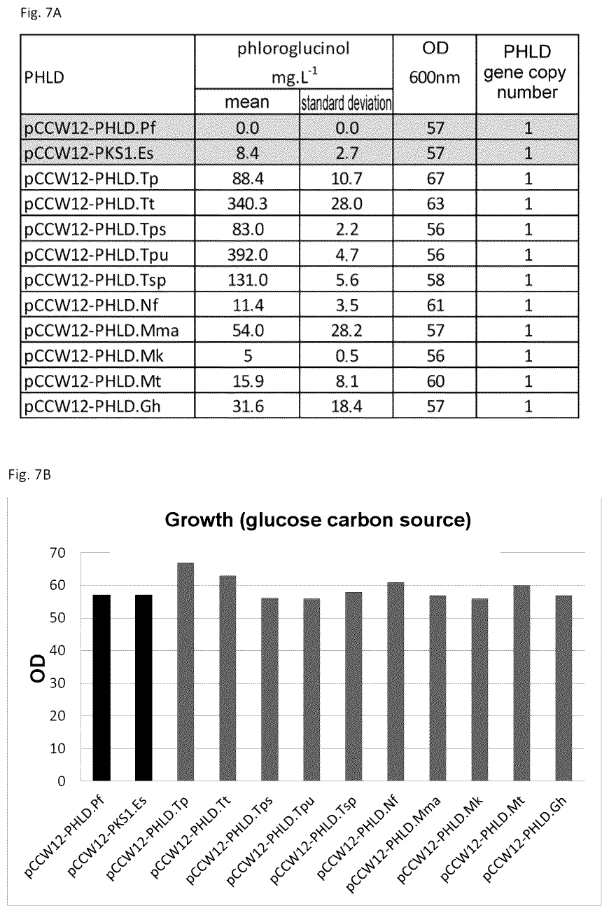

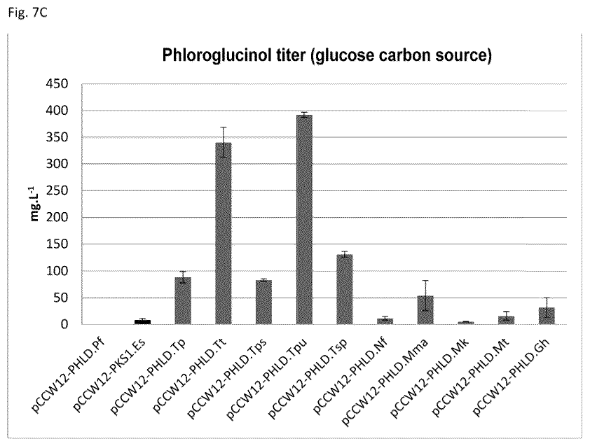

The yeast strains expressing a single copy of PHLD or of PKS1 were cultured in the presence of 20 g.l⁻¹ of ethanol as carbon source (constructs controlled by pADH2) or in the presence of 20 g.l⁻¹ of glucose (constructs controlled by pCCW12) for 48 hours at 30° C. (FIGS. 6 and 7). The strains were cultured and analysed for their capacity to produce phloroglucinol.

The optical densities of the various cultures were measured at 600 nm (indicating the level of growth of each strain, FIGS. 6A and B and FIGS. 7A and B) and the phloroglucinol production (in mg.l⁻¹) was measured in the culture medium, for 2 independent transformants for each construct (FIGS. 6A and C and FIGS. 7A and 9C).

FIG. 6 (A and C) shows that the strains expressing a single copy of the PHLD.Tp, PHLD.Tt, PHLD.Tps, PHLD.Tpu, PHLD.Tsp, PHLD.Nf, PHLD.Mma, PHLD.Mk, PHLD.Mt or PHLD.Gh genes under the control of the ADH2 promoter synthesize a measurable and significant amount of phloroglucinol which is excreted into the culture medium, in the presence of ethanol as carbon source.

For the cultures carried out in the presence of glucose as carbon source, the strains expressing a single copy of the PHLD.Tp, PHLD.Tt, PHLD.Tps, PHLD.Tpu, PHLD.Tsp, PHLD.Nf, PHLD.Mma, PHLD.Mk, PHLD.Mt and PHLD.Gh genes under the control of the CCW12 promoter all synthesize phloroglucinol, in an amount that is comparable to the cultures carried out in the presence of ethanol as carbon source (FIGS. 7A and C).

The results obtained above confirm the results obtained in the strains containing several copies of the phloroglucinol synthases described in section 2.1 above. Thus, the expression of the PHLD.Tp, PHLD.Tt, PHLD.Tps, PHLD.Tpu, PHLD.Tsp, PHLD.Nf, PHLD.Mma, PHLD.Mk, PHLD.Mt and PHLD.Gh candidate enzymes results in the significant production of phloroglucinol by the yeast cells. These results show that these genes encode phloroglucinol synthases and that these phloroglucinol synthases are active in yeast cells.

These results additionally show that the levels of phloroglucinol production are high even when a single copy of the gene encoding the phloroglucinol synthase is expressed.

These results are the first demonstrations of the existence of a phloroglucinol synthase activity in the gram+ type bacteria.

More generally, the results show that the PHLD.ii candidate enzymes tested exhibit an activity that overall is higher than the only two phloroglucinol synthases known before this study, i.e. the enzymes of E. siliculosus and P. fluorescens. The PHLD candidate enzymes of the Tsukamurella genus (PHLD.Tt, PHLD.Tps, PHLD.Tpu, PHLD.Tsp, PHLD.Tp) are particularly active when they are expressed in yeast. Indeed, the phloroglucinol production standardized by the number of copies demonstrates that the Tsukamurella enzymes tested (PHLD.Tp, PHLD.Tt, PHLD.Tpu, PHLD.Tps and PHLD.Tsp) have a phloroglucinol synthase activity that is at least 10 times higher than that of the PKS1 enzyme of Ectocarpus siliculosus in the expression system in yeast tested herein. According to these results, the PHLD.Tpu enzyme appears in particular to be the most active enzyme, under the various conditions tested.

The 3 enzymes PHLD.Mma (of Mycobacterium marinum), PHLD.Nf (of Nocardia farcinica) and PHLD.Gh (of Gordonia hydrophobica) are also significantly very active.

The present invention thus provides several new original and advantageous pathways for the biosynthesis of phloroglucinol in yeast.

### 2.3. Additional Observations

The study carried out by the inventors and reported here made it possible to identify 10 new enzymes having a phloroglucinol synthase activity. These 10 new phloroglucinol synthases come from gram+ bacteria of the order Actinomycetales, including the Tsukamurella genus (PHLD.Tt, PHLD.Tps, PHLD.Tpu, PHLD.Tsp, PHLD.Tp), the Nocardia genus (PHLD.Nf); the Mycobacterium genus (PHLD.Mma, PHLD.Mt and PHLD.Mk) and the Gordonia genus (PHLD.Gh) and have never been described up until now as producing phloroglucinol.

These results therefore demonstrate for the first time and unequivocally that it is possible to synthesize phloroglucinol in yeast cells.

It is important to note that the phloroglucinol synthesized is more than 95% secreted into the culture medium by the yeast cells. This particularly efficient secretion is very favourable to the implementation of a phloroglucinol bioproduction process.

The phloroglucinol synthases of the actinomycete bacteria of the Tsukamurella genus appear to be the most active according to the results obtained to date. Indeed, the results obtained show that the PHLD enzymes of Tsukamurella sp., expressed in yeast cells, are at least 10 times more active than the phloroglucinol synthase of the brown alga Ectocarpus siliculosus of the prior art, under the conditions tested.

The PHLD enzymes of Tsukamurella sp. could thus represent enzymes of choice for implementing a process of phloroglucinol bioproduction in the yeast S. cerevisiae.

Finally, the enzymes identified by the inventors are original in terms of species of origin and in terms of protein sequences. Indeed, it is shown for the first time that Gram+ bacteria encode functional phloroglucinol synthases. In addition, the protein sequence of the enzyme that is the most active under the experimental conditions tested by the inventors, PHLD.Tpu of Tsukamurella pulmonis, differs significantly from the sequence of the known PKS1.Es enzyme of Ectocarpus siliculosus (difference of more than 63%, Table 3).

### LITERATURE REFERENCES

Achkar J et al., (2005) “Biosynthesis of phloroglucinol” J Am Chem Soc. 127: 5332-5333.
Meslet-Cladière L, Delage L, Leroux CJ, Goulitquer S, Leblanc C, Creis E, Gall EA, Stiger-Pouvreau V, Czjzek M, and Potin P. (2013) “Structure/function analysis of a type III polyketide synthase in the brown alga Ectocarpus siliculosus reveals a biochemical pathway in phlorotannin monomer biosynthesis.” Plant Cell. 25: 3089-3103.
Zha W, Rubin-Pitel SB and Zhao H. (2006) “Characterization of the substrate specificity of PHLD, a type III polyketide synthase from Pseudomonas fluorescens.” J Biol Chem. 281: 32036-32047.

1.-17. (canceled)

18. An isolated nucleic acid molecule comprising:
at least one nucleic acid sequence encoding a polypeptide selected from type III polyketide synthases of bacteria; and
an exogenous promoter controlling expression of the at least one nucleic acid sequence,
wherein the type III polyketide synthases of bacteria exclude type III polyketide synthase of Pseudomonas fluorescens PHLD.Pf.

19. The isolated nucleic acid molecule according to claim 18, wherein the exogenous promoter is a yeast promoter.

20. The isolated nucleic acid molecule according to claim 18, further comprising a transcription terminator for the at least one nucleic acid sequence.

21. A vector comprising the isolated nucleic acid molecule according to claim 18.

22. A host cell comprising at least one nucleic acid molecule or comprising a vector comprising the at least one nucleic acid molecule, wherein the at least one nucleic acid molecule is the isolated nucleic acid molecule according to claim 18.

23. The host cell according to claim 22, wherein the host cell is a microorganism selected from the group consisting of bacteria, yeast, fungi, algae and cyanobacteria.

24. The host cell according to claim 22, wherein at least one copy of the at least one nucleic acid molecule is integrated into a genome of the host cell.

25. An isolated nucleic acid molecule comprising:
at least one nucleic acid sequence encoding a polypeptide selected from type III polyketide synthases of bacteria; and
an exogenous terminator controlling expression of the at least one nucleic acid sequence,
wherein the type III polyketide synthases of bacteria exclude type III polyketide synthase of Pseudomonas fluorescens PHLD.Pf.

26. The isolated nucleic acid molecule according to claim 25, wherein the exogenous terminator is a yeast terminator.

27. The isolated nucleic acid molecule according to claim 25, further comprising a promoter controlling expression of the at least one nucleic acid sequence.

28. A vector comprising the isolated nucleic acid molecule according to claim 25.

29. A host cell comprising at least one nucleic acid molecule or comprising a vector comprising the at least one nucleic acid molecule, wherein the at least one nucleic acid molecule is the isolated nucleic acid molecule according to claim 25.

30. The host cell according to claim 29, wherein the host cell is a microorganism selected from the group consisting of bacteria, yeast, fungi, algae and cyanobacteria.

31. The host cell according to claim 29, wherein at least one copy of the at least one nucleic acid molecule is integrated into a genome of the host cell.

32. An isolated nucleic acid molecule comprising:
at least one nucleic acid sequence encoding a polypeptide selected from type III polyketide synthases of bacteria;
a yeast promoter controlling expression of the at least one nucleic acid sequence; and
a yeast terminator controlling the expression of the at least one nucleic acid sequence,
wherein the type III polyketide synthases of bacteria exclude type III polyketide synthase of Pseudomonas fluorescens PHLD.Pf.

33. A vector comprising the isolated nucleic acid molecule according to claim 32.

34. A host cell comprising at least one nucleic acid molecule or comprising a vector comprising the at least one nucleic acid molecule, wherein the at least one nucleic acid molecule is the isolated nucleic acid molecule according to claim 32.

35. The host cell according to claim 34, wherein the host cell is a microorganism selected from the group consisting of bacteria, yeast, fungi, algae and cyanobacteria.

36. The host cell according to claim 34, wherein at least one copy of the at least one nucleic acid molecule is integrated into a genome of the host cell.
